(19) United States
(12) Patent Application Publication
(10) Pub. No.: US 2025/0041069 A1
(43) Pub. Date: Feb. 6, 2025

(54) Fixation Methods for Total Ankle Arthroplasty

(71) Applicant: Arthrex, Inc., Naples, FL (US)

(72) Inventors: Adam N. Garlock, Naples, FL (US); Chris Powell, Naples, FL (US); Braham Dhillon, Naples, FL (US); Maris Prieditis, Naples, FL (US); Benjamin Chan, Bonita Springs, FL (US)

(21) Appl. No.: 18/714,183

(22) PCT Filed: Dec. 2, 2022
(86) PCT No.: PCT/US2022/051676

§ 371 Date: May 29, 2024

Related U.S. Application Data
(60) Provisional application No. 63/285,827, filed on Dec. 3, 2021.
(60) Provisional application No. 63/286,713, filed on Dec. 7, 2021.

(51) Int. Cl. A61F 2/42; A61F 2/30; A61F 2/46

(52) U.S. Cl. CPC A61F 2/4202; A61F 2/4606; A61F 2002/30092; A61F 2002/30235; A61F 2002/30476; A61F 2002/30545; A61F 2002/4205; A61F 2002/4207

(57) ABSTRACT

Total ankle replacement (TAR) requires fixation of the tibial component to the tibia of a patient. and further requires fixation of the talar component to the talar of the patient. Disclosed herein are prosthetic implants that provide a high retention force to aid in preventing loosening after initial implantation. The prosthetic implant includes an expanding peg or post that allows the surgeon to expand the peg intraoperatively to apply radial pressure resulting in a wedged press fit that provides enhanced time zero fixation. As such. the present disclosure provides multiple fixation methods that allow for increased fixation during initial implantation of both the tibial and talar components in TAR surgery by providing dynamic compression between the prosthetic implant and the mating bone so that the prosthetic implant does not move or migrate after implantation.

### CROSS-REFERNECE TO RELATED APPLICATIONS

This application claims the benefit of priority to (i) U.S. Provisional Application No. 63/285,827 entitled “Fixation Methods for Total Ankle Arthroplasty,” filed on Dec. 3, 2021, and (ii) U.S. Provisional Application No. 63/286,713 entitled “Dynamic Fixation Methods for Total Ankle Replacement,” filed on Dec. 7, 2021, the contents of each of which are hereby incorporated by reference in their entirety.

### BACKGROUND

Total ankle replacement (TAR) requires fixation of the tibial component to the tibia of a patient, and further requires fixation of the talar component to the talar of the patient. Disclosed herein are prosthetic implants that provide a high retention force to aid in preventing loosening after initial implantation.

### SUMMARY

The present disclosure includes a prosthetic implant for use in TAR surgery. The prosthetic implant of the present disclosure includes an expanding peg or post that allows the surgeon to expand the peg intraoperatively to apply radial pressure resulting in a wedged press fit that provides enhanced time zero fixation. As such, the present disclosure provides multiple fixation methods that allow for increased fixation during initial implantation of both the tibial and talar components in TAR surgery by providing dynamic compression between the prosthetic implant and the mating bone so that the prosthetic implant does not move or migrate after implantation.

The present disclosure provides one or more pegs or posts of the prosthetic implant are secured to the bone of the patient by a press fit between a prepared hole in the bone and the outer diameter of the peg/post. Further, the present disclosure allows the surgeon to apply radial pressure by expanding the peg or post to obtain robust fixation in addition to the press fit. Once the peg or post is positioned in the prepared hole in the bone, an expanding mechanism of the peg or post is moved from a first position to a second position to thereby cause the peg or post to expand either outward in a tapered direction or radially causing the peg to have a mechanical press fit with the mating bone. As such, the present disclosure provides increased fixation during initial implantation until the body has time to heal and osseointegration with the porous ingrowth surface occurs.

Thus, in one aspect, a prosthetic implant includes a body comprising a first side and a second side opposite the first side. The first side comprises a bone contact surface, and the second side is configured to be coupled to an articulation surface. The prosthetic implant also includes a cylindrical stem extending away from the first side of the body. The prosthetic implant also includes an expanding mechanism positioned within the cylindrical stem. Movement of the expanding mechanism from a first position to a second position causes a maximum diameter of the cylindrical stem to change from a first maximum diameter to a second maximum diameter, where the second maximum diameter is greater than the first maximum diameter.

In another aspect, a method can include drilling a channel in a bone, positioning a cylindrical stem of a prosthetic implant into the channel, where the prosthetic implant includes a body comprising a first side and a second side opposite the first side, where the cylindrical stem extends away from the first side, and where the second side is configured to be coupled to an articulation surface, and moving an expanding mechanism positioned within the cylindrical stem from a first position to a second position, thereby causing a maximum diameter of the cylindrical stem to change from a first maximum diameter to a second maximum diameter, where the second maximum diameter is greater than the first maximum diameter.

In another aspect, a prosthetic implant includes a body comprising a first side and a second side opposite the first side, where the first side comprises a bone contact surface and the second side is configured to be coupled to an articulation surface. The prosthetic implant further includes a cylindrical stem spaced away from the first side of the body. The prosthetic implant further includes a rod having a first end and a second end opposite the first end. The first end is coupled to the first side of the body and the second end is coupled to the cylindrical stem. The rod comprises a shape memory or super elastic material. The rod is stretched prior to placement of the cylindrical stem in a channel in a tibia of a patient such that the shape memory or super elastic material of the rod causes the cylindrical stem and the body to be pulled towards each other.

In another aspect, a prosthetic implant includes a body comprising a first side and a second side opposite the first side, where the first side comprises a bone contact surface and the second side is configured to be coupled to an articulation surface. The prosthetic implant further includes a cylindrical stem extending away from the first side of the body. The prosthetic implant further includes a shape memory or super elastic component positioned at least partially within the cylindrical stem. The prosthetic implant further includes a holding mechanism positioned coupled to the shape memory or super elastic component. Removal of the holding mechanism causes the shape memory or super elastic component to retract from a first length to a second length, where the first length is greater than the second length. Retraction of the shape memory or super elastic component from the first length to the second length causes a maximum diameter of the cylindrical stem to change from a first maximum diameter to a second maximum diameter, where the second maximum diameter is greater than the first maximum diameter.

In another aspect, a prosthetic implant includes a base plate including a first side and a second side opposite the first side. The prosthetic implant further includes an outer core extending away from the first side of the base plate. The outer core is configured to be positioned within a bone. The prosthetic implant further includes an inner core coupled to the base plate and positioned at least partially within the outer core. The prosthetic implant further includes an attachment mechanism coupling the inner core to the outer core. The attachment mechanism allows longitudinal movement of the inner core with respect to the outer core in a first direction while preventing longitudinal movement of the inner core with respect to the outer core in a second direction opposite the first direction.

### BRIEF DESCRIPTION OF THE DRAWINGS

FIG. 1 is a perspective view of an example prosthetic device.

FIG. 2 is a side cross-sectional view of the example prosthetic device of FIG. 1.

FIG. 3 is a perspective view of another example prosthetic device.

FIG. 4 is a side cross-sectional view of the example prosthetic device of FIG. 3.

FIG. 5 is a perspective view of another example prosthetic device.

FIG. 6 is a top view of the example prosthetic device of FIG. 5.

FIG. 7 is a perspective view of another example prosthetic device.

FIG. 8 is a perspective view of another example prosthetic device.

FIG. 9 is a side cross-sectional view of another example prosthetic device.

FIG. 10 is a side cross-sectional view of another example prosthetic device.

FIG. 11 is a side cross-sectional view of another example prosthetic device.

FIG. 12 is a perspective view of an example prosthetic implant.

FIG. 13 is a side cross-sectional view of another example prosthetic implant.

FIG. 14 is a side cross-sectional view of another example prosthetic implant.

FIG. 15 is a side cross-sectional view of one end of the example prosthetic implant of FIG. 14.

FIG. 16 is a side cross-sectional view of a shape memory or super elastic component and a holding mechanism of the example prosthetic implant of FIG. 14.

FIG. 17 is a side cross-sectional view of another example prosthetic implant.

FIG. 18 is a side cross-sectional view of the prosthetic implant of FIG. 17 in a compressed state.

FIG. 19 is a side cross-sectional view of another example prosthetic implant.

FIG. 20 is a side cross-sectional view of another example prosthetic implant.

### DETAILED DESCRIPTION

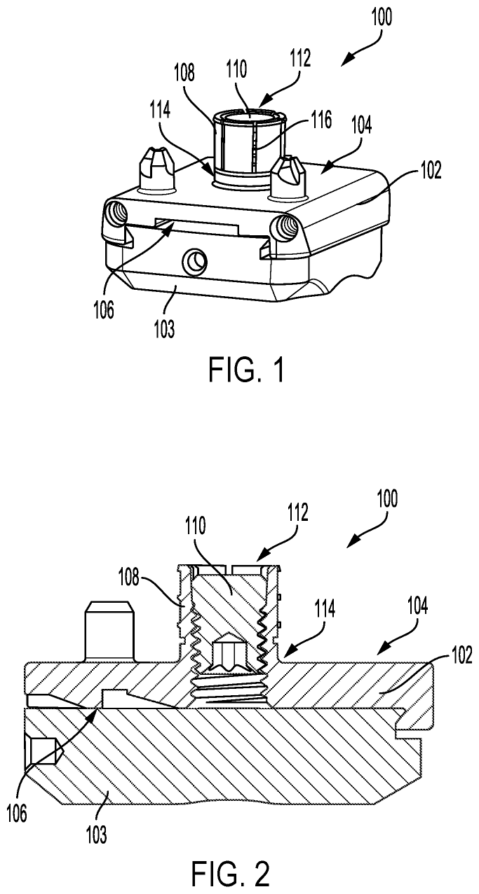

With reference to the Figures, FIG. 1 illustrates a prosthetic implant 100 including a body 102 comprising a first side 104 and a second side 106 opposite the first side 104. The first side 104 comprises a bone contact surface, and the second side 106 is configured to be coupled to an articulation surface 103. The prosthetic implant 100 further includes a cylindrical stem 108 extending away from the first side 104 of the body 102. The prosthetic implant 100 further includes an expanding mechanism 110 positioned within the cylindrical stem 108. Movement of the expanding mechanism 110 from a first position to a second position causes a maximum diameter of the cylindrical stem 108 to change from a first maximum diameter to a second maximum diameter, where the second maximum diameter is greater than the first maximum diameter. The expanding mechanism can be a set screw.

In an example, as shown in FIGS. 1-2, a longitudinal axis of the cylindrical stem 108 is perpendicular to the first side 104 of the body 102. The cylindrical stem 108 has a first end 112 and a second end 114 opposite the first end 112, and the second end 114 of the cylindrical stem 108 is directly coupled to the first side 104 of the body 102. The cylindrical stem 108 can include a plurality of slits 116 extending from the first end 112 in a direction towards the second end 114. The expanding mechanism 110 can be accessed from the second side 106 of the body 102. The expanding mechanism 110 can move in a direction towards the second side 106 of the body 102 from the first position to the second position to thereby cause the cylindrical stem 108 to change from the first maximum diameter to the second maximum diameter.

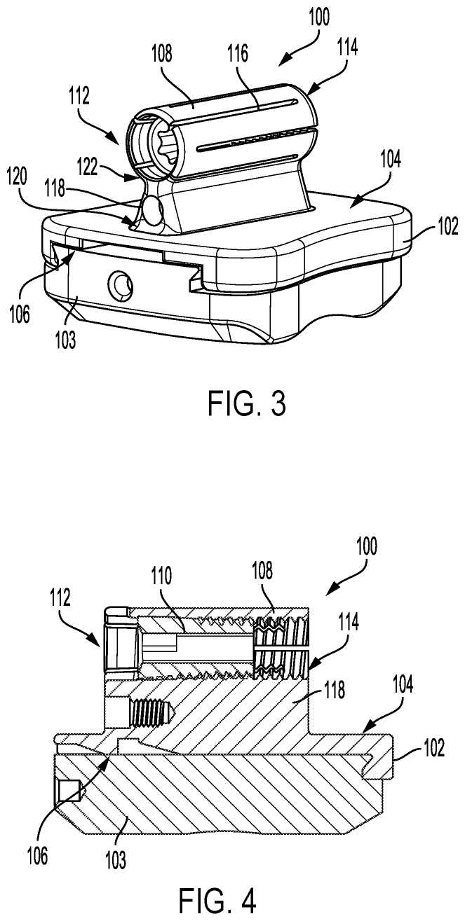

In an example, as shown in FIGS. 3-4, a longitudinal axis of the cylindrical stem 108 is parallel to the first side 104 of the body 102. The prosthetic implant 100 may further include a longitudinal support 118 having a first end 120 and a second end 122 opposite the first end 120. The first end 120 is coupled to the first side 104 of the body 102, and the second end 122 is coupled to the cylindrical stem 108 such that the cylindrical stem 108 is spaced away from the body 102. As discussed above, the cylindrical stem 108 has a first end 112 and a second end 114 opposite the first end 112. A diameter of the first end 112 and the diameter of the second end 114 can remain constant when the expanding mechanism 110 moves from the first position to the second position such that the maximum diameter of the cylindrical stem 108 occurs between the first end 112 and the second end 114. Alternatively, the maximum diameter of the cylindrical stem can occur at either the first end 112 or the second end 114.

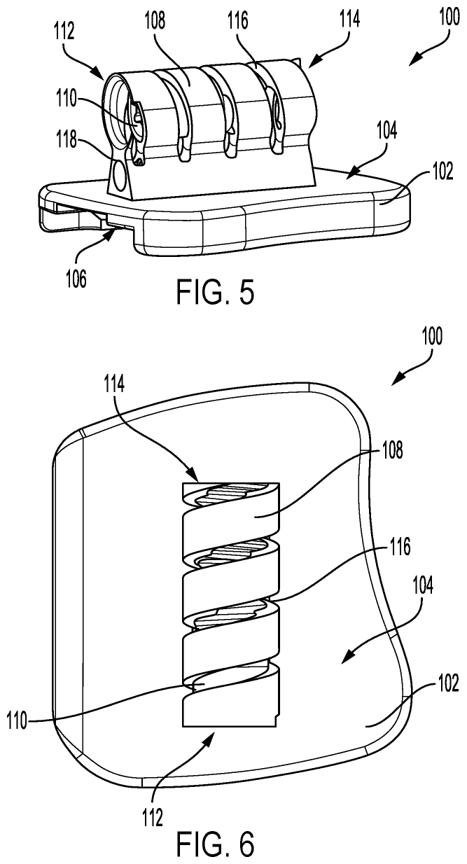

In an example, the cylindrical stem 108 includes a plurality of slits 116 between the first end 112 and the second end 114. The plurality of slits 116 may include a first plurality of slits extending from the first end 112 in a direction towards the second end 114 and includes a second plurality of slits extending from the second end 114 in a direction towards the first end 112. The plurality of slits 116 can be substantially parallel with the longitudinal axis of the cylindrical stem 108, as shown in FIG. 3. As shown in FIGS. 5-6, the plurality of slits 116 can form a helical pattern around the cylindrical stem 108.

In use, in one example, the cylindrical stem 108 is configured to be positioned within a channel formed in a tibia of a patient. In another example, the cylindrical stem 108 is configured to be positioned within a channel formed in a talus of a patient.

The prosthetic implant 100 can further include a second cylindrical stem extending away from the first side 104 of the body 102, and a second expanding mechanism positioned within the second cylindrical stem. Similar to the expanding mechanism 110 described above, movement of the second expanding mechanism from a first position to a second position causes a maximum diameter of the second cylindrical stem to change from a first maximum diameter to a second maximum diameter, where the second maximum diameter is greater than the first maximum diameter.

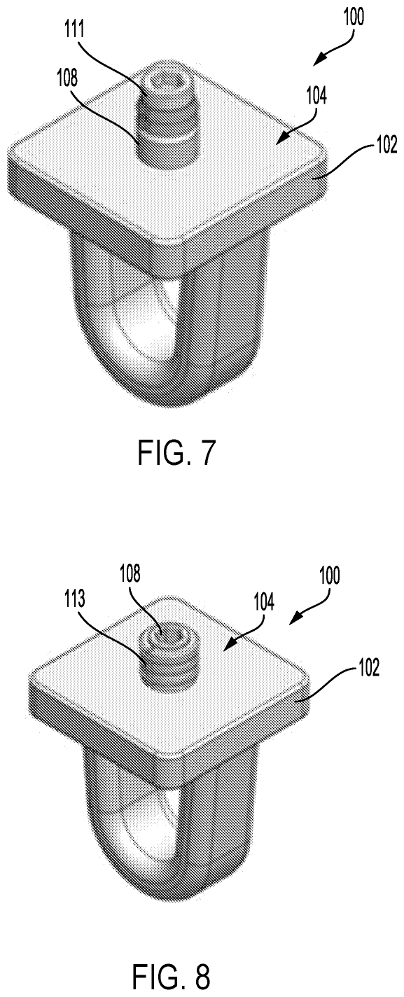

In an example, as shown in FIG. 7, the expanding mechanism 110 comprises a down biting collet 111. In such an example, the down biting collet 111 may be positioned on an exterior surface of the cylindrical stem 108, and may be configured to expand based on a vertical movement of the cylindrical stem 108 into a hole or channel formed in bone (e.g., a tibia or talus) of a patient.

In another example, as shown in FIG. 8, the expanding mechanism 110 comprises an expanding collet 113. In such an example, the expanding collet 113 may be positioned on an exterior surface of the cylindrical stem 108, and may be configured to expand based on a vertical movement of the cylindrical stem 108 into a hole or channel formed in bone (e.g., a tibia or talus) of a patient.

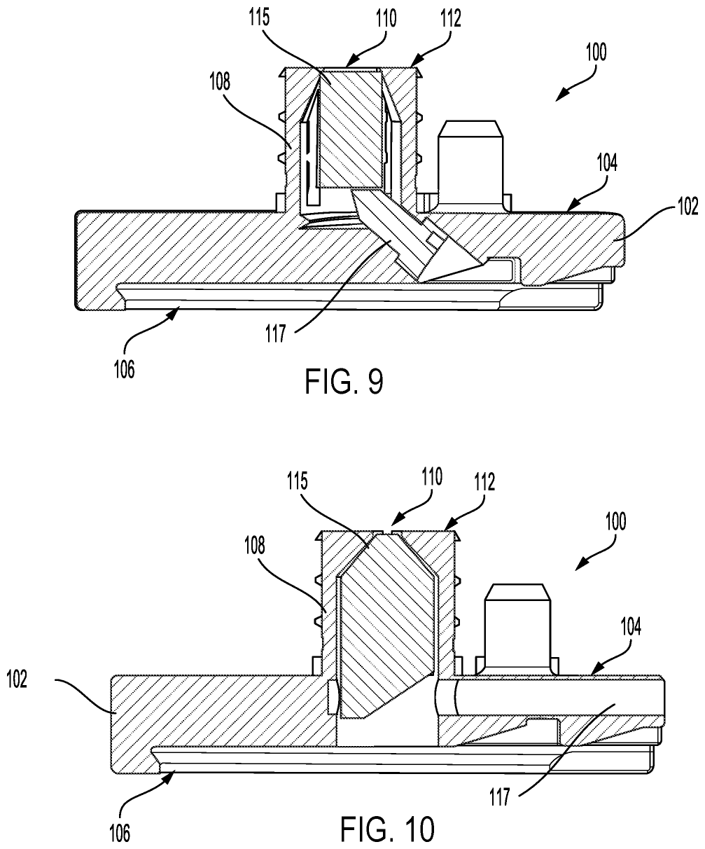

In another example, as shown in FIGS. 9-11, the expanding mechanism 110 includes an elongated member 115 positioned in the cylindrical stem 108, and a contact member 117 configured to contact a bottom surface of the elongated member 115 to move the elongated member 115 towards the first end 112 of the cylindrical stem 108 to thereby transition the expanding mechanism 110 from the first position to the second position.

In one example, as shown in FIG. 9, the elongated member 115 is cylindrical in shape and contacts an angled surface of the interior of the cylindrical stem 108 to thereby transition the expanding mechanism 110 from the first position to the second position. In one example, as shown in FIG. 9, the contact member 117 comprises an angled screw that is configured to contact a bottom surface of the elongated member 115 to move the elongated member 115 towards the first end 112 of the cylindrical stem 108.

In another example, as shown in FIG. 10, the elongated member 115 is cylindrical in shape with a tapered end that contacts an angled surface of the interior of the cylindrical stem 108 to thereby transition the expanding mechanism 110 from the first position to the second position. In one example, as shown in FIG. 9, the contact member 117 comprises a rod that is configured to contact an angled bottom surface of the elongated member 115 to move the elongated member 115 towards the first end 112 of the cylindrical stem 108.

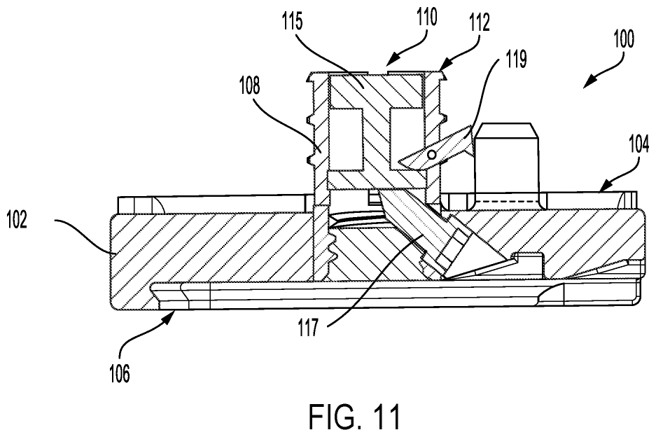

In another example, as shown in FIG. 11, the prosthetic implant 100 further includes a lever 119 configured to contact the elongated member 115 to thereby lock the expanding mechanism 110 in the second position. In one such example, the lever 119 is positioned in a first position that allows movement of the elongated member 115 towards the first end 112 of the cylindrical stem 108. Once the expanding mechanism 110 has transitioned to the second position, the lever 119 moves to a second position (as shown in FIG. 11) to thereby lock the expanding mechanism 110 in the second position.

Methods disclosed herein can be used with any of the embodiments of the prosthetic implant 100 as described herein.

A method includes fixing a prosthetic implant 100 to a bone of a patient. A method can include drilling a channel in a bone. In an example, the channel does not extend entirely through the bone. A method can also include positioning a cylindrical stem 108 of the prosthetic implant 100 into the channel. The prosthetic implant 100 includes a body 102 comprising a first side 104 and a second side 106 opposite the first side 104, the cylindrical stem 108 extends away from the first side 104, and the second side 106 is configured to be coupled to an articulation surface 103. A method can also include moving an expanding mechanism 110 positioned within the cylindrical stem 108 from a first position to a second position, thereby causing a maximum diameter of the cylindrical stem 108 to change from a first maximum diameter to a second maximum diameter, were the second maximum diameter is greater than the first maximum diameter.

In an example, a longitudinal axis of the cylindrical stem 108 is perpendicular to the first side 104 of the body 102. The expanding mechanism 110 can be accessed from the second side 106 of the body 102. The expanding mechanism 110 can move in a direction towards the second side 106 of the body 102 from the first position to the second position to thereby cause the cylindrical stem 108 to change from the first maximum diameter to the second maximum diameter.

In an example, a longitudinal axis of the cylindrical stem 108 is parallel to the first side 104 of the body 102. A diameter of the first end 112 of the cylindrical stem 108 and the diameter of the second end 114 of the cylindrical stem 108 can remain constant when the expanding mechanism 110 moves from the first position to the second position. The maximum diameter of the cylindrical stem can occur between the first end 112 and the second end 114. Alternatively, the maximum diameter of the cylindrical stem 108 can occur at either the first end 112 or at the second end 114.

In an example, the channel is drilled in a tibia of a patient or in a talus of a patient.

In an example, a method further includes drilling a second channel in the bone, positioning a second cylindrical stem of the prosthetic implant into the second channel, where the second cylindrical stem extends away from the first side 104 of the body 102 of the prosthetic implant 100, and moving a second expanding mechanism positioned within the second cylindrical stem from a first position to a second position, thereby causing a maximum diameter of the second cylindrical stem to change from a first maximum diameter to a second maximum diameter, where the second maximum diameter is greater than the first maximum diameter.

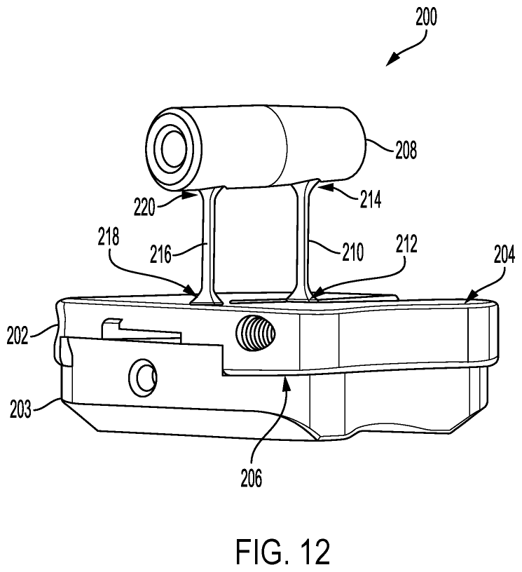

FIG. 12 illustrates another prosthetic implant 200 including a body 202 comprising a first side 204 and a second side 206 opposite the first side 204. The first side 204 comprises a bone contact surface, and the second side 206 is configured to be coupled to an articulation surface 203. The prosthetic implant 200 further includes a cylindrical stem 208 spaced away from the first side 204 of the body 202. In an example, a longitudinal axis of the cylindrical stem 208 is parallel to the first side 204 of the body 202. The prosthetic implant 200 further includes a rod 210 having a first end 212 and a second end 214 opposite the first end 212. The first end 212 of the rod 210 is coupled to the first side 204 of the body 202, and the second end 214 of the rod 210 is coupled to the cylindrical stem 208. The rod 210 comprises a shape memory or super elastic material. In an example, the shape memory or super elastic material of the rod 210 comprises a nickel-titanium alloy. The rod 210 is stretched prior to placement of the cylindrical stem 208 in a channel in a tibia of a patient such that the shape memory or super elastic material of the rod 210 causes the cylindrical stem 208 and the body 202 to be pulled towards each other after implantation. This shortening of the rod 210 results in constant compression between the prosthetic implant 200 and the tibia, thus reducing the likelihood of implant loosening or subsidence. In an example, the rod 210 is stretched intraoperatively prior to placement of the cylindrical stem 208 in the channel in the tibia. In another example, the rod 210 is pre-stretched prior to placement of the cylindrical stem 208 in the channel in the tibia.

In an example, as shown in FIG. 12, the prosthetic implant 200 can include a second rod 216 similarly configured to the rod 210. In an example, the second rod 216 includes a first end 218 and a second end 220 opposite the first end 218. The first end 218 of the second rod 216 is coupled to the first side 104 of the body 202, and the second end 220 of the second rod 216 is coupled to the cylindrical stem 208. The second rod 216 comprises a shape memory or super elastic material. The second rod 216 is stretched prior to placement of the cylindrical stem 208 in a channel in a tibia of a patient such that the shape memory or super elastic material of the second rod 216 causes the cylindrical stem 208 and the body 202 to be pulled towards each other after implantation.

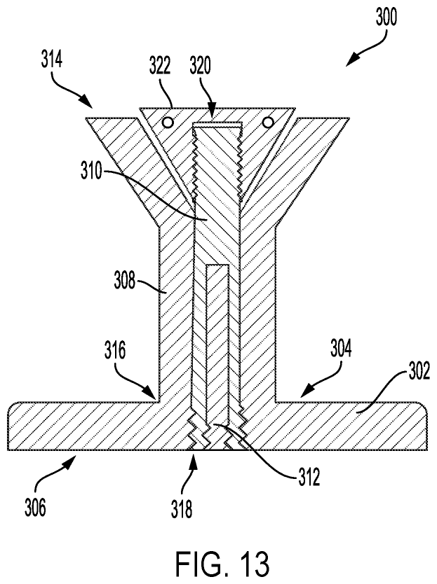

FIG. 13 illustrates another example prosthetic implant 300 including a body 302 comprising a first side 304 and a second side 306 opposite the first side 304. The first side 304 comprises a bone contact surface, and the second side 306 is configured to be coupled to an articulation surface 303. The prosthetic implant 300 further includes a cylindrical stem 308 extending away from the first side 304 of the body 302. The prosthetic implant 300 further includes a shape memory or super elastic component 310 positioned at least partially within the cylindrical stem 308. In an example, the shape memory or super elastic component 310 comprises a nickel-titanium alloy. The prosthetic implant 300 further includes a holding mechanism 312 positioned coupled to the shape memory or super elastic component 310. Removal of the holding mechanism 312 causes the shape memory or super elastic component 310 to retract from a first length to a second length, where the first length is greater than the second length. Retraction of the shape memory or super elastic component 310 from the first length to the second length causes a maximum diameter of the cylindrical stem 308 to change from a first maximum diameter to a second maximum diameter, where the second maximum diameter is greater than the first maximum diameter.

In an example, as shown in FIG. 13, a longitudinal axis of the cylindrical stem 308 is perpendicular to the first side 304 of the body 302. In one such example, the cylindrical stem 308 has a first end 314 and a second end 316 opposite the first end 314, and the second end 316 of the cylindrical stem 308 is directly coupled to the first side 304 of the body 302. In an example, the holding mechanism 312 is accessed from the second side 306 of the body 302. In an example, the cylindrical stem 308 includes a plurality of slits extending from the first end 314 in a direction towards the second end 316. The plurality of slits can help enable the cylindrical stem 308 to change from the first maximum diameter to the second maximum diameter as the shape memory or super elastic component 310 retracts from the first length to the second length.

In an example, as shown in FIG. 13, the shape memory or super elastic component 310 has a first end 318 and a second end 320 opposite the first end 318, and the holding mechanism 312 is coupled to the first end 318 of the shape memory or super elastic component 310. An opening wedge 322 can be coupled to the second end 320 of the shape memory or super elastic component 310, such that retraction of the shape memory or super elastic component 310 from the first length to the second length causes the opening wedge 322 to move towards the first end 318 of the shape memory or super elastic component 310 to thereby cause the maximum diameter of the cylindrical stem 308 to change from the first maximum diameter to the second maximum diameter.

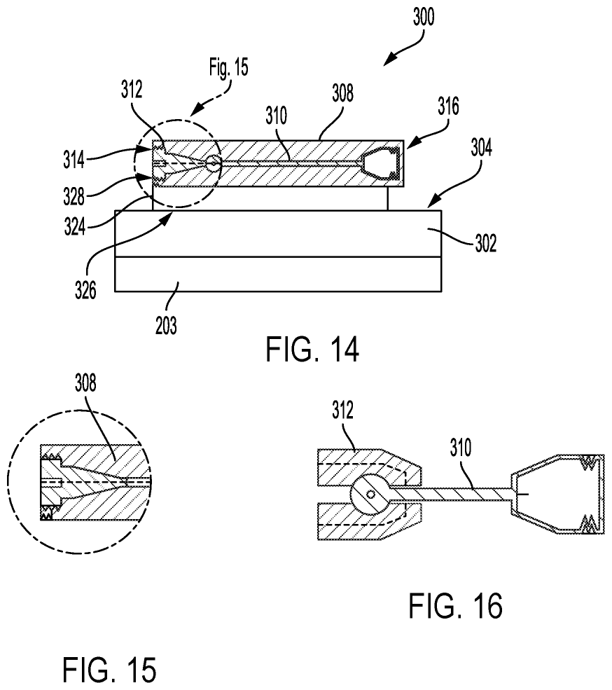

In an example, as shown in FIGS. 14-16, a longitudinal axis of the cylindrical stem 308 is parallel to the first side 304 of the body 302. In such an example, the prosthetic implant 300 can include a longitudinal support 324 having a first end 326 and a second end 328 opposite the first end 326. The first end 326 of the longitudinal support 324 is coupled to the first side 304 of the body 302, and the second end 328 of the longitudinal support 324 is coupled to the cylindrical stem 308 such that the cylindrical stem 308 is spaced away from the body 302.

In an example, the maximum diameter of the cylindrical stem 308 occurs between the first end 314 and the second end 316 of the cylindrical stem 308. In one such example, a diameter of the first end 314 of the cylindrical stem 208 and a diameter of the second end 316 of the cylindrical stem 308 remain constant when the holding mechanism 312 retracts from the first length to the second length. In an example, the cylindrical stem 308 includes a plurality of slits positioned between the first end 314 and the second end 316. The plurality of slits can help enable the cylindrical stem 308 to change from the first maximum diameter to the second maximum diameter as the shape memory or super elastic component 310 retracts from the first length to the second length.

In an example, the first end 318 of the shape memory or super elastic component 310 is fixedly coupled to the cylindrical stem 308, and the second end 320 of the shape memory or super elastic component 310 is coupled to an opening wedge 322. In such an example, retraction of the shape memory or super elastic component 310 from the first length to the second length causes the opening wedge 322 to move towards the first end 318 of the shape memory or super elastic component 310 to thereby cause the maximum diameter of the cylindrical stem 208 to change from the first maximum diameter to the second maximum diameter.

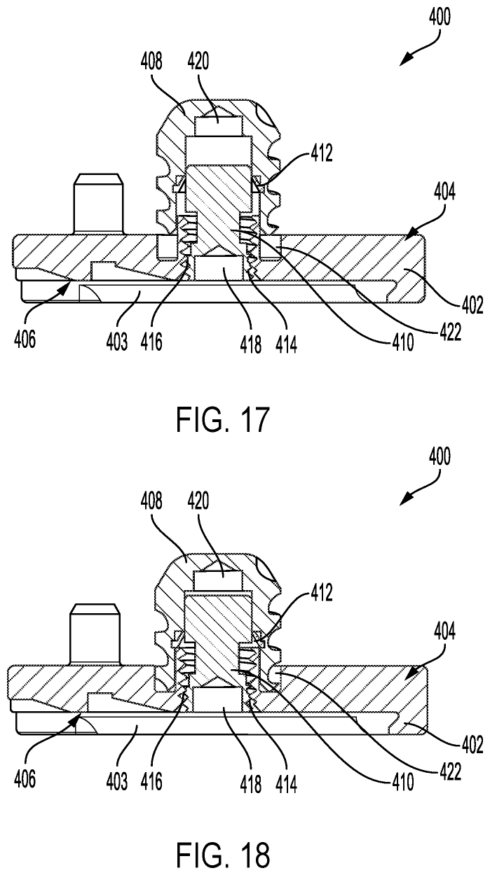

FIG. 17 illustrates another example prosthetic implant 400 including a base plate 402 including a first side 404 and a second side 406 opposite the first side 404. The prosthetic implant 400 further includes an outer core 408 extending away from the first side 404 of the base plate 402. The outer core 408 is configured to be positioned within a bone of a patient. The prosthetic implant 400 further includes an inner core 410 coupled to the base plate 402 and positioned at least partially within the outer core 408. The prosthetic implant 400 further includes an attachment mechanism 412 coupling the inner core 410 to the outer core 408. The attachment mechanism 412 allows longitudinal movement of the inner core 410 with respect to the outer core 408 in a first direction while preventing longitudinal movement of the inner core 410 with respect to the outer core 408 in a second direction opposite the first direction. In an example, the base plate 402 and the inner core 410 are coupled to one another, and as loading during gait occurs the base plate 402 and the inner core 410 move in an upward direction (towards the bone) while the outer core 408 stays stationary in the bone. The attachment mechanism 412 prevents the base plate 402 and inner core 410 from moving in the opposite direction (away from the bone).

In an example, as shown in FIGS. 17-18, an exterior surface of the inner core 410 includes a first plurality of threads 414, and a portion of the base plate 402 includes a second plurality of threads 416 configured to interact with the first plurality of threads 414 such that the inner core 410 is able to move rotationally and independently from the outer core 408. During a revision surgery, the articulation surface 403 is removed from the base plate 402 exposing the driver mechanism 418 for the inner core 410. As discussed above, the inner core 410 is able to move rotationally, and independently, from the outer core 408 via the first plurality of threads 414 and the second plurality of threads 416. This independent movement allows the inner core 410 to overcome the force of the attachment mechanism 412 to release the base plate 402 and inner core 410 from the outer core 408, which is affixed to the tibia of the patient. Once the base plate 402 and inner core 410 are removed, the internal driver mechanism 420 at the top of the outer core 408 is exposed, which allows the surgeon to unthread the outer core 408 from the tibial bone.

In an example, as shown in FIGS. 17-18, the first side 404 of the base plate 402 includes a channel 422, and a portion of the outer core 408 is configured to nest within the channel 422 as the prosthetic implant 400 compresses. FIG. 17 illustrates the starting position of the inner core 410 and outer core 408 relative to one another. As weight bearing begins, and bone resorption is present, compressive forces allow the inner core 410 to piston causing the base plate 402 to move upward. As the base plate 402 moves up relative to the outer core 408, the channel 422 on the top surface of the base plate 402 allows for the outer core 408 to nest within the base plate 402, which is shown in FIG. 18.

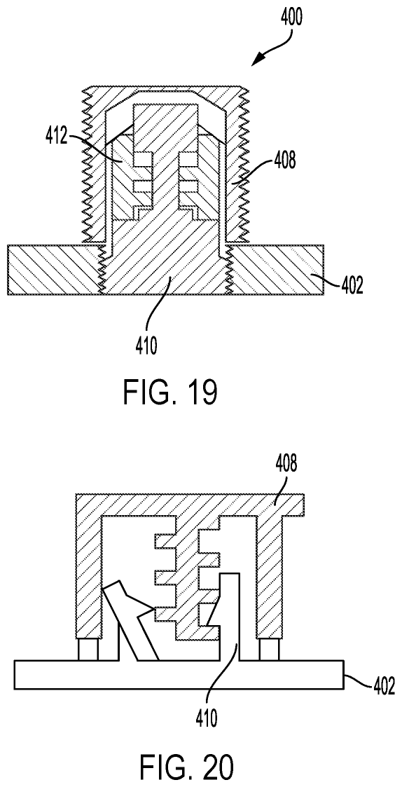

In an example, as shown in FIGS. 19-20, the attachment mechanism 412 comprises a grab ring. In an example, as shown in FIG. 19, the outer core 408 and inner core 410 include flange/undercut features to retain the attachment mechanism 412. In another example, as shown in FIG. 20, the attachment mechanism 412 comprises a ratchet mechanism, and an inner surface of the inner core 410 includes a plurality of protrusions configured to interact with the ratchet mechanism.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and examples have been disclosed herein, other aspects and examples will be apparent to those skilled in the art. The various aspects and examples disclosed herein are for purposes of illustration and are not intended to be limiting, with the true scope being indicated by the following claims, along with the full scope of equivalents to which such claims are entitled. It is also to be understood that the terminology used herein is for the purpose of describing particular examples only, and is not intended to be limiting.

Example methods and systems are described herein. It should be understood that the words “example,” “exemplary,” and “illustrative” are used herein to mean “serving as an example, instance, or illustration.” Any example or feature described herein as being an “example,” being “exemplary,” or being “illustrative” is not necessarily to be construed as preferred or advantageous over other examples or features. The examples described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other examples may include more or less of each element shown in a given Figure. Further, some of the illustrated elements may be combined or omitted. Yet further, an example may include elements that are not illustrated in the Figures.

In the following description, numerous specific details are set forth to provide a thorough understanding of the disclosed concepts, which may be practiced without some or all of these particulars. In other instances, details of known devices and/or processes have been omitted to avoid unnecessarily obscuring the disclosure. While some concepts will be described in conjunction with specific examples, it will be understood that these examples are not intended to be limiting.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

The limitations of the following claims are not written in means-plus-function format and are not intended to be interpreted based on 35 U.S.C. § 112 (f), unless and until such claim limitations expressly use the phrase “means for” followed by a statement of function void of further structure.

By the term “about,” “approximately,” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide. For example, in one embodiment, the term “about” can refer to ±5% of a given value.

Illustrative, non-exhaustive examples, which may or may not be claimed, of the subject matter according the present disclosure are provided below.

What is claimed is:

1. A prosthetic implant comprising:
a body comprising a first side and a second side opposite the first side, wherein the first side comprises a bone contact surface, and wherein the second side is configured to be coupled to an articulation surface;
a cylindrical stem extending away from the first side of the body; and
an expanding mechanism positioned within the cylindrical stem, wherein movement of the expanding mechanism from a first position to a second position causes a maximum diameter of the cylindrical stem to change from a first maximum diameter to a second maximum diameter, and wherein the second maximum diameter is greater than the first maximum diameter.

2. The prosthetic implant of claim 1, wherein a longitudinal axis of the cylindrical stem is perpendicular to the first side of the body.

3. The prosthetic implant of claim 2, wherein the cylindrical stem has a first end and a second end opposite the first end, and wherein the second end of the cylindrical stem is directly coupled to the first side of the body.

4. The prosthetic implant of any one of claims 2-3, wherein the cylindrical stem has a first end and a second end opposite the first end, and wherein the cylindrical stem includes a plurality of slits extending from the first end in a direction towards the second end.

5. The prosthetic implant of any one of claims 2-4, wherein the expanding mechanism is accessed from the second side of the body, and wherein the expanding mechanism moves in a direction towards the second side of the body from the first position to the second position to thereby cause the cylindrical stem to change from the first maximum diameter to the second maximum diameter.

6. The prosthetic implant of claim 1, wherein a longitudinal axis of the cylindrical stem is parallel to the first side of the body.

7. The prosthetic implant of claim 6, further comprising a longitudinal support having a first end and a second end opposite the first end, wherein the first end is coupled to the first side of the body, and wherein the second end is coupled to the cylindrical stem such that the cylindrical stem is spaced away from the body.

8. The prosthetic implant of any one of claims 6-7, wherein the cylindrical stem has a first end and a second end opposite the first end.

9. The prosthetic implant of claim 8, wherein a diameter of the first end and the diameter of the second end remain constant when the expanding mechanism moves from the first position to the second position, and wherein the maximum diameter of the cylindrical stem occurs between the first end and the second end.

10. The prosthetic implant of claim 8, wherein the maximum diameter of the cylindrical stem occurs at either the first end or the second end.

11. The prosthetic implant of any one of claims 6-8, wherein the cylindrical stem has a first end and a second end opposite the first end, wherein the cylindrical stem includes a plurality of slits between the first end and the second end.

12. The prosthetic implant of claim 11, wherein the plurality of slits are substantially parallel with the longitudinal axis of the cylindrical stem.

13. The prosthetic implant of claim 11, wherein the plurality of slits form a helical pattern around the cylindrical stem.

14. The prosthetic implant of any one of claims 1-13, wherein the cylindrical stem is configured to be positioned within a channel formed in a tibia of a patient.

15. The prosthetic implant of any one of claims 1-13, wherein the cylindrical stem is configured to be positioned within a channel formed in a talus of a patient.

16. The prosthetic implant of any one of claims 1-15, further comprising:
a second cylindrical stem extending away from the first side of the body; and
a second expanding mechanism positioned within the second cylindrical stem, wherein movement of the second expanding mechanism from a first position to a second position causes a maximum diameter of the second cylindrical stem to change from a first maximum diameter to a second maximum diameter, and wherein the second maximum diameter is greater than the first maximum diameter.

17. The prosthetic implant of any one of claims 1-16, wherein the expanding mechanism comprises a set screw.

18. The prosthetic implant of any one of claims 1-17, wherein the expanding mechanism comprises a down biting collet.

19. The prosthetic implant of any one of claims 1-18, wherein the expanding mechanism comprises an expanding collet.

20. The prosthetic implant of any one of claims 1-19, wherein the expanding mechanism comprises:
an elongated member positioned in the cylindrical stem; and
a contact member configured to contact a bottom surface of the elongated member to move the elongated member towards a first end of the cylindrical stem to thereby transition the expanding mechanism from the first position to the second position.

21. The prosthetic implant of claim 20, further comprising:
a lever configured to contact the elongated member to thereby lock the expanding mechanism in the second position.

22. A method comprising:
drilling a channel in a bone;
positioning a cylindrical stem of a prosthetic implant into the channel, wherein the prosthetic implant includes a body comprising a first side and a second side opposite the first side, wherein the cylindrical stem extends away from the first side, and wherein the second side is configured to be coupled to an articulation surface; and
moving an expanding mechanism positioned within the cylindrical stem from a first position to a second position, thereby causing a maximum diameter of the cylindrical stem to change from a first maximum diameter to a second maximum diameter, wherein the second maximum diameter is greater than the first maximum diameter.

23. The method of claim 22, wherein the channel does not extend entirely through the bone.

24. The method of any one of claims 22-23, wherein a longitudinal axis of the cylindrical stem is perpendicular to the first side of the body, wherein the expanding mechanism is accessed from the second side of the body, and wherein the expanding mechanism moves in a direction towards the second side of the body from the first position to the second position to thereby cause the cylindrical stem to change from the first maximum diameter to the second maximum diameter.

25. The method of any one of claims 22-23, wherein a longitudinal axis of the cylindrical stem is parallel to the first side of the body, wherein the cylindrical stem has a first end and a second end opposite the first end.

26. The method of claim 25, wherein a diameter of the first end and the diameter of the second end remain constant when the expanding mechanism moves from the first position to the second position, and wherein the maximum diameter of the cylindrical stem occurs between the first end and the second end.

27. The method of claim 25, wherein the maximum diameter of the cylindrical stem occurs at either the first end or the second end.

28. The method of any one of claims 22-27, wherein the channel is drilled in a tibia of a patient.

29. The method of any one of claims 22-27, wherein the channel is drilled in a talus of a patient.

30. The method of any one of claims 22-29, further comprising:
drilling a second channel in the bone;
positioning a second cylindrical stem of the prosthetic implant into the second channel, wherein the second cylindrical stem extends away from the first side of the body of the prosthetic implant; and
moving a second expanding mechanism positioned within the second cylindrical stem from a first position to a second position, thereby causing a maximum diameter of the second cylindrical stem to change from a first maximum diameter to a second maximum diameter, wherein the second maximum diameter is greater than the first maximum diameter.

31. A prosthetic implant comprising:
a body comprising a first side and a second side opposite the first side, wherein the first side comprises a bone contact surface, and wherein the second side is configured to be coupled to an articulation surface;
a cylindrical stem spaced away from the first side of the body; and
a rod having a first end and a second end opposite the first end, wherein the first end is coupled to the first side of the body, wherein the second end is coupled to the cylindrical stem, wherein the rod comprises a shape memory or super elastic material, and wherein the rod is stretched prior to placement of the cylindrical stem in a channel in a tibia of a patient such that the shape memory or super elastic material of the rod causes the cylindrical stem and the body to be pulled towards each other.

32. The prosthetic implant of claim 31, wherein the shape memory or super elastic material of the rod comprises a nickel-titanium alloy.

33. The prosthetic implant of any one of claims 31-32, further comprising:
a second rod having a first end and a second end opposite the first end, wherein the first end of the second rod is coupled to the first side of the body, wherein the second end of the second rod is coupled to the cylindrical stem, wherein the second rod comprises a shape memory or super elastic material, and wherein the second rod is stretched prior to placement of the cylindrical stem in a channel in a tibia of a patient such that the shape memory or super elastic material of the second rod causes the cylindrical stem and the body to be pulled towards each other.

34. The prosthetic implant of any one of claims 31-33, wherein a longitudinal axis of the cylindrical stem is parallel to the first side of the body.

35. The prosthetic implant of any one of claims 31-34, wherein the rod is stretched intraoperatively prior to placement of the cylindrical stem in the channel in the tibia.

36. A prosthetic implant comprising:
a body comprising a first side and a second side opposite the first side, wherein the first side comprises a bone contact surface, and wherein the second side is configured to be coupled to an articulation surface;
a cylindrical stem extending away from the first side of the body;
a shape memory or super elastic component positioned at least partially within the cylindrical stem; and
a holding mechanism positioned coupled to the shape memory or super elastic component, wherein removal of the holding mechanism causes the shape memory or super elastic component to retract from a first length to a second length, wherein the first length is greater than the second length, wherein retraction of the shape memory or super elastic component from the first length to the second length causes a maximum diameter of the cylindrical stem to change from a first maximum diameter to a second maximum diameter, and wherein the second maximum diameter is greater than the first maximum diameter.

37. The prosthetic implant of claim 36, wherein a longitudinal axis of the cylindrical stem is perpendicular to the first side of the body.

38. The prosthetic implant of claim 37, wherein the cylindrical stem has a first end and a second end opposite the first end, and wherein the second end of the cylindrical stem is directly coupled to the first side of the body.

39. The prosthetic implant of any one of claims 36-38, wherein the holding mechanism is accessed from the second side of the body.

40. The prosthetic implant of any one of claims 36-39, wherein the shape memory or super elastic component has a first end and a second end opposite the first end, wherein the holding mechanism is coupled to the first end of the shape memory or super elastic component, wherein an opening wedge is coupled to the second end of the shape memory or super elastic component, and wherein retraction of the shape memory or super elastic component from the first length to the second length causes the opening wedge to move towards the first end of the shape memory or super elastic component to thereby cause the maximum diameter of the cylindrical stem to change from the first maximum diameter to the second maximum diameter.

41. The prosthetic implant of any one of claims 36-40, wherein the cylindrical stem has a first end and a second end opposite the first end, and wherein the cylindrical stem includes a plurality of slits extending from the first end in a direction towards the second end.

42. The prosthetic implant of any one of claims 36-41, wherein a longitudinal axis of the cylindrical stem is parallel to the first side of the body.

43. The prosthetic implant of claim 42, further comprising a longitudinal support having a first end and a second end opposite the first end, wherein the first end is coupled to the first side of the body, and wherein the second end is coupled to the cylindrical stem such that the cylindrical stem is spaced away from the body.

44. The prosthetic implant of any one of claims 42-43, wherein the cylindrical stem has a first end and a second end opposite the first end, wherein the maximum diameter of the cylindrical stem occurs between the first end and the second end.

45. The prosthetic implant of any one of claims 42-44, wherein the cylindrical stem has a first end and a second end opposite the first end, wherein the cylindrical stem includes a plurality of slits positioned between the first end and the second end of the cylindrical stem.

46. The prosthetic implant of any one of claims 42-45, wherein the shape memory or super elastic component includes a first end and a second end opposite the first end, wherein the first end of the shape memory or super elastic component is fixedly coupled to the cylindrical stem, and wherein the second end of the shape memory or super elastic component is coupled to an opening wedge, and wherein retraction of the shape memory or super elastic component from the first length to the second length causes the opening wedge to move towards the first end of the shape memory or super elastic component to thereby cause the maximum diameter of the cylindrical stem to change from the first maximum diameter to the second maximum diameter.

47. The prosthetic implant of any one of claims 36-46, wherein the shape memory or super elastic component comprises a nickel-titanium alloy.

48. A prosthetic implant comprising:
a base plate including a first side and a second side opposite the first side;
an outer core extending away from the first side of the base plate, wherein the outer core is configured to be positioned within a bone; and
an inner core coupled to the base plate and positioned at least partially within the outer core; and
an attachment mechanism coupling the inner core to the outer core, wherein the attachment mechanism allows longitudinal movement of the inner core with respect to the outer core in a first direction while preventing longitudinal movement of the inner core with respect to the outer core in a second direction opposite the first direction.

49. The prosthetic implant of claim 48, wherein the attachment mechanism comprises a grab ring.

50. The prosthetic implant of claim 48, wherein the attachment mechanism comprises a ratchet mechanism, and wherein an inner surface of the inner core includes a plurality of protrusions configured to interact with the ratchet mechanism.

51. The prosthetic implant of any one of claims 48-50, wherein an exterior surface of the inner core includes a first plurality of threads, and wherein a portion of the base plate includes a second plurality of threads configured to interact with the first plurality of threads such that the inner core is able to move rotationally and independently from the outer core.

52. The prosthetic implant of any one of claims 48-51, wherein the first side of the base plate includes a channel, and wherein a portion of the outer core is configured to nest within the channel as the prosthetic implant compresses.
